(19) United States
(12) Patent Application Publication
(10) Pub. No.: US 2025/0090390 A1
(43) Pub. Date: Mar. 20, 2025

(54) Feminine Hygiene Product Removal Device

(71) Applicant: Tamara Atkins, Secane, PA (US)

(72) Inventor: Tamara Atkins, Secane, PA (US)

(21) Appl. No.: 18/368,129

(22) Filed: Sep. 14, 2023

Related U.S. Application Data
(60) Provisional application No. 63/389,297, filed on Jul. 14, 2022.

(51) Int. Cl. A61F 13/551

(52) U.S. Cl. CPC A61F 13/5515

(57) ABSTRACT

A used feminine hygiene product disposal pad includes an outer cover, a middle absorbent layer, an inner containment layer, and a drawstring component. The outer cover is held by the user when grasping the used feminine hygiene product, the middle absorbent layer absorbs moisture or fluid from the used feminine hygiene product, and the inner containment layer comes into direct contact with the used feminine hygiene product being grasped and wraps around the hygiene product. The drawstring component is used to securely tie the disposal pad around the used feminine hygiene product.

### FIELD OF THE INVENTION

The present invention relates to the field of feminine hygiene. Specifically, the present invention relates to a method and device that assists in quickly and easily allowing the user to remove and contain a used feminine hygiene product, for discreet and clean disposal of the used product.

### CROSS-REFERENCE TO RELATED APPLICATIONS

The present application claims priority from provisional application 63/389,297 filed on Jul. 14, 2022, the contents of which are hereby incorporated by reference into this application.

### BACKGROUND

Traditional feminine hygiene product disposal methods and devices, are unable to sufficiently remove and dispose of used feminine hygiene products through a collective disposal unit. This restriction often makes it difficult to effectively and discreetly remove and discard used feminine hygiene products.

Prior techniques and devices used for the previous scenario, do not effectively provide solutions to this problem, as they consist of insubstantial and vulnerable materials and methods of use, that are susceptible to mechanical and physical inabilities, and limiting device capabilities and capacities.

Therefore, what is needed are techniques that overcome the above-mentioned disadvantages and problems.

### BRIEF SUMMARY

The present invention, in several embodiments, is, but not limited to, a device and method that quickly and easily allows a user to remove and contain a used feminine hygiene product, for clean and discreet disposal. The present invention provides this solution through its novel and unique design and features.

In a preferred embodiment, the present invention consists of a circular collective tampon removal pad comprising of an outer cover layer, a middle absorbent layer, an inner containment layer, and a drawstring component. The outer cover layer, which preferably is, but not limited to, consists of a plastic lining to protect the user from the used tampon. The middle absorbent layer preferably consists of, but is not limited to, a thin cotton lining to absorb any excess moisture from the used tampon. The inner containment layer preferably consists of, but is not limited to, a thin deodorizer lining which is where the used tampon is to be contained. All three layers of the tampon disposal pad work collectively to remove and contain the used tampon. The drawstring component is contained around the edges of the disposal pad. Once the used tampon is contained within the disposal pad, the user can use the drawstring component to tightly wrap and secure the used tampon in place within the disposal pad. Once the used tampon is secured within the disposal pad, the user can discreetly dispose of the used tampon, while maintaining a high level of sanitation and cleanliness.

In accordance with an additional embodiment of the present invention, a method of using the present invention is provided as described in the Detailed Description section below. The Detailed Description section also provides a variety of additional aspects, features, and descriptions.

### BRIEF DESCRIPTION OF THE SEVERAL VIEWS OF THE DRAWINGS

The present invention is illustrated by way of example and not limitation in the figures of the accompanying drawings in which like references indicate similar elements.

Furthermore, the present invention, and all its embodiments, will be more fully understood and comprehended through the elements in the Detailed Description in conjunction with the accompanying drawing(s).

FIG. 1 illustrates a top perspective view of the disposal pad, according to one embodiment of the present invention.

FIG. 2 illustrates a top perspective view of the disposal pad showing the middle absorbent layer in accordance with one embodiment.

FIG. 3 illustrates a top perspective view of the disposal pad showing the inner containment layer in accordance with one embodiment.

FIG. 4A illustrates a bottom perspective view of the disposal pad in accordance with one embodiment.

FIG. 4B illustrates a cross-sectional view of the disposal pad in accordance with one embodiment.

FIG. 5 illustrates a top view of the disposal pad showing a folding method in accordance with one embodiment.

FIG. 6 illustrates a top view of the disposal pad showing more of the folding method in accordance with one embodiment.

FIG. 7 illustrates a top view of the disposal pad showing more of the folding method in accordance with one embodiment.

FIG. 8 illustrates a top view of the disposal pad folded and tied in accordance with one embodiment.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

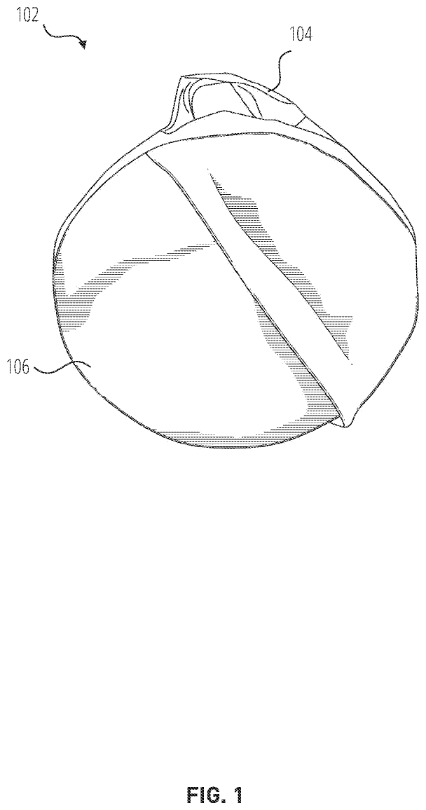

FIG. 1 illustrates the perspective of an embodiment from the top view of the present invention. This embodiment of the present invention shows feminine hygiene product disposal pad (hereinafter “the disposal pad”) 102 comprised of an outer cover 106, a middle absorbent layer 206, an inner containment layer 306, and a drawstring component 104. The outer cover 106 is the protective layer in which the user will grasp to remove the used tampon. The absorbent layer 206 is the subsequent layer which will absorb any excess fluid. The inner containment layer 306 is the bottom layer of the disposal pad. This bottom layer is the layer to physically contact and contain the used tampon. The inner containment layer 306 preferably consists of a deodorizer lining. The drawstring component 104 is incorporated around the edges of the disposal pad, and used to securely tie the used tampon within the disposal pad.

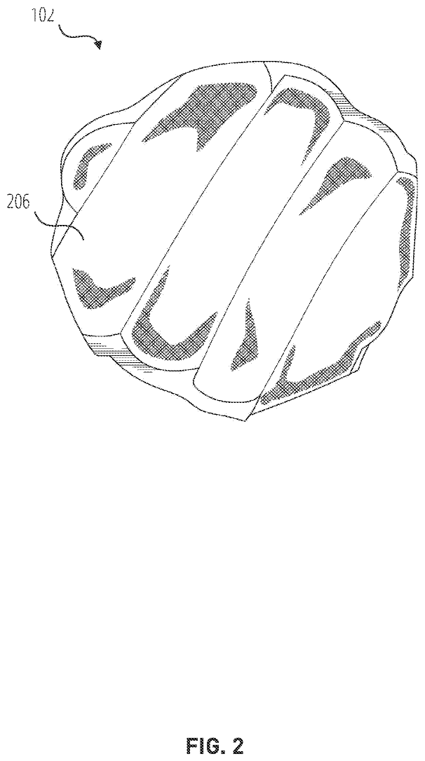

FIG. 2 illustrates the diagram of an embodiment from the top view of the present invention. This embodiment of the present invention is comprised of all the same elements and features in FIG. 1. All aspects and elements outlined in FIG. 1 are applicable to FIG. 2. This embodiment of the present invention illustrates the middle absorbent layer 206 of the disposal pad 102. This layer is securely positioned between the outer cover 106 and inner containment layer 306 of the present invention.

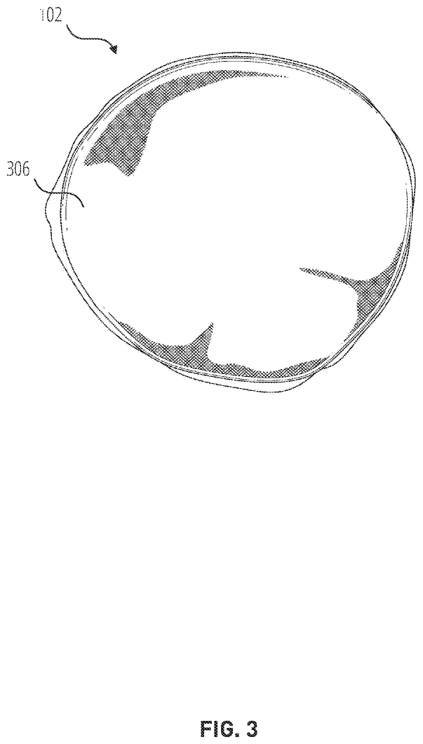

FIG. 3 illustrates the diagram of an embodiment from the top view of the present invention. This embodiment of the present invention is comprised of all the same elements and features in FIG. 1. All aspects and elements outlined in FIG. 1 are applicable to FIG. 3. This embodiment of the present invention illustrates the inner containment layer 306 of the disposal pad. This layer is securely positioned as the bottom layer of the present invention.

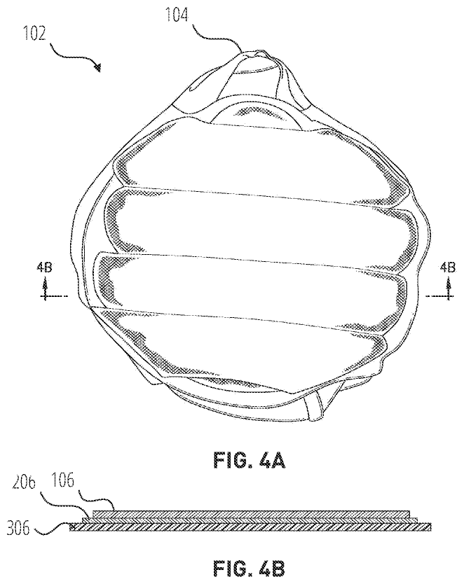

FIG. 4A illustrates the diagram of an embodiment from the bottom view of the present invention. This embodiment of the present invention is comprised of all the same elements and features in FIG. 1. All aspects and elements outlined in FIG. 1 are applicable to FIG. 4.

FIG. 4B illustrates a cross-sectional view of the disposal pad along lines 4B-4B showing the different layers, i.e., the outer cover 106, the middle absorbent layer 206, and the inner containment layer 306 in accordance with an embodiment of the present invention.

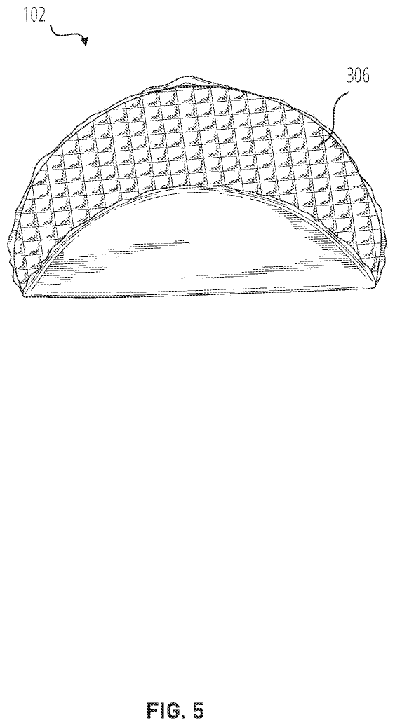

FIG. 5 illustrates the diagram of an embodiment from the top view of the present invention. This embodiment of the present invention is comprised of all the same elements and features in FIG. 1. All aspects and elements outlined in FIG. 1 are applicable to FIG. 5. This embodiment of the present invention is to illustrate a folding method of the disposal pad, to contain a used tampon within.

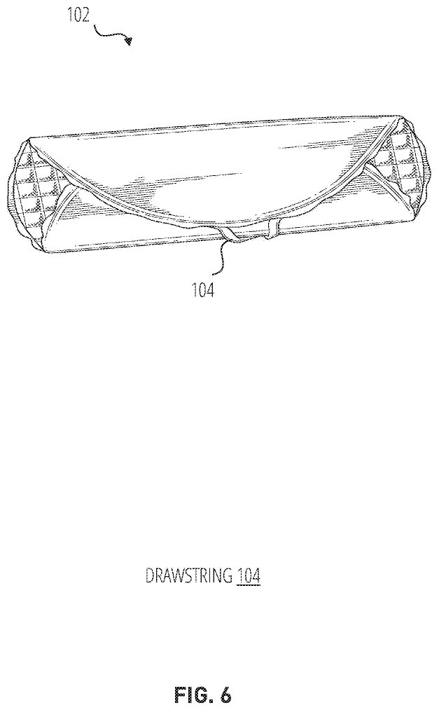

FIG. 6 illustrates the diagram of an embodiment from the top view of the present invention. This embodiment of the present invention is comprised of all the same elements and features in FIGS. 1 and 5. All aspects and elements outlined in FIGS. 1 and 5 are applicable to FIG. 6. FIG. 6 shows more of the folding method.

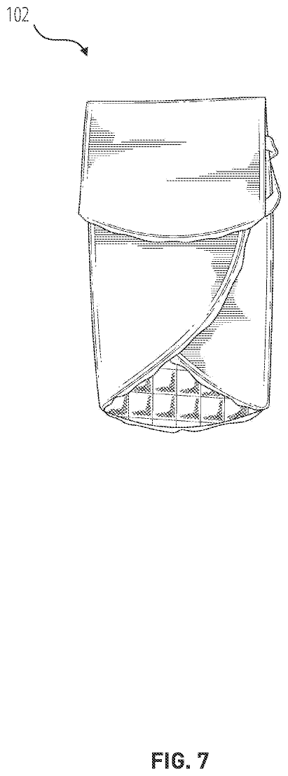

FIG. 7 illustrates the diagram of an embodiment from the top view of the present invention. This embodiment of the present invention is comprised of all the same elements and features in FIGS. 1 and 5. All aspects and elements outlined in FIGS. 1 and 5 are applicable to FIG. 7. FIG. 7 shows more of the folding method.

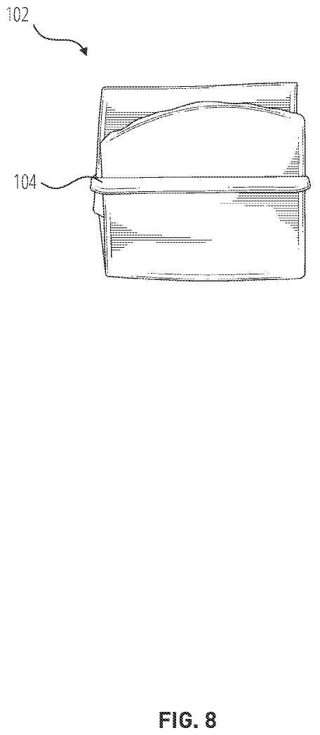

FIG. 8 illustrates the diagram of an embodiment from the top view of the present invention. This embodiment of the present invention is comprised of all the same elements and features in FIGS. 1 and 5. All aspects and elements outlined in FIGS. 1 and 5 are applicable to FIG. 8. FIG. 8 shows the disposal pad folded and tied with drawstring 104.

Although the figures depict a round shaped disposal pad, the disposal pad may have other shapes, such as square, rectangle, triangle, ellipse, or polygon without departing from the spirit or scope of the invention. For example, a square shaped disposal pad with all the features described above may be used instead of a round disposal pad.

In addition, the disposal pad of the present invention may take the form of a mitten or glove with the same features described above. In one embodiment, the mitten or glove is a plastic glove covered with an exterior absorbent material that prevents a user's hand from getting soiled.

All elements of all embodiments, as previous specified and/or unspecified, of the present invention, are easily and capably produced through various manufacturing techniques. All elements of all embodiments, as previous specified and/or unspecified, of the present invention can be manufactured at various lengths, widths, heights, thicknesses, weights, designs, shapes, sizes, colors, and other various features and characteristics. The present invention can be manufactured from various types of materials and compositions, including various metals and/or metal alloys, various plastics and/or plastic compositions, various woods, and/or other various organic and/or inorganic compounds and materials.

The preferred embodiment of the present invention utilizes an outer cover consisting of a thin plastic lining, a middle layer consisting of an absorbent lining, an inner containment layer consisting of a deodorizer lining, and a drawstring component. The present invention may also utilize various methods and materials of implementing each element within the present invention.

As demonstrated from all descriptions and illustrations outlined above and below, it is clearly evident that the present invention, and all of its embodiments, is an effective device and method for, but not limited to, quickly and easily removing and securing a used feminine hygiene product through a collective disposal unit, which allows discreet and clean disposal of the product. It is evident that the present invention, and all of its embodiments, provides these effective functions as outlined and disclosed, due to its novel, unique, and advantageous designs, characteristics, and features.

Although the descriptions and illustrations of this disclosure contains many specifications, these should not be interpreted as limiting the scope of the present invention and all its embodiments, but rather providing demonstrations of some of the various embodiments. Various modifications and alterations of this disclosure will become apparent to those skilled in the art without departing from the scope and spirit of this disclosure, and it should be understood that the scope of this disclosure is not to be unduly limited to the illustrative embodiments set forth herein.

What is claimed is:

1. A used feminine hygiene product disposal pad comprising:
an outer cover,
a middle absorbent layer,
an inner containment layer, and
a drawstring component,
wherein the outer cover is held by the user when grasping the used feminine hygiene product, the middle absorbent layer absorbs moisture or fluid from the used feminine hygiene product, the inner containment layer comes into direct contact with the used feminine hygiene product and wraps around the hygiene product, and the drawstring component is used to securely tie the disposal pad around the used feminine hygiene product.

2. The feminine hygiene disposal pad of claim 1, wherein the outer cover, middle absorbent layer, and inner containment layer collectively form a barrier between a user's hand and the used feminine hygiene product when the user grasps the used feminine hygiene product and, after grasping the used feminine hygiene product, the disposal pad is folded around the used hygiene product and tied with the drawstring for subsequent disposal of the hygiene product.

3. The feminine hygiene disposal pad of claim 1, wherein the inner containment layer comprises a deodorizer lining.

4. The feminine hygiene disposal pad of claim 1, wherein the drawstring component is disposed around an edge of the disposal pad.

5. The feminine hygiene disposal pad of claim 1, wherein the disposal pad is in the form of a mitten.

6. A method for disposing of a used feminine hygiene product comprising:
holding a disposal pad comprising:
an outer cover,
a middle absorbent layer that absorbs moisture or fluid from the hygiene product,
an inner containment layer that comes into direct contact with the hygiene product, and
a drawstring component,
wherein the disposal pad is held by its outer cover so that the inner containment layer is facing the used feminine hygiene product;
grasping the used feminine hygiene product and folding the disposal pad around the used feminine hygiene product;
pulling the drawstring and tying it around the folded disposal pad with the used feminine hygiene product enclosed therein;
disposing of the disposal pad with the used feminine hygiene product inside.

7. The method of claim 6, wherein the inner containment layer comprises a deodorizer lining.

8. The method of claim 6, wherein the drawstring component is disposed around an edge of the disposal pad.

9. The method of claim 6, wherein the disposal pad is in the form of a mitten.
